(19) United States
(12) Plant Patent Application Publication
(10) Pub. No.: US 2013/0239268 P1
(43) Pub. Date: Sep. 12, 2013

(54) Eucalyptus tree named 'G5'

(50) ;

(71) Applicant: Donald L. Rockwood, Gainesville, FL (US)

(72) Inventor: Donald L. Rockwood, Gainesville, FL (US)

(73) Assignee: Florida Foundation Seed Producers, Inc., Marianna, FL (US)

(21) Appl. No.: 13/385,867

(22) Filed: Mar. 9, 2012

(51) Int. Cl. A01H 5/00

(52) U.S. Cl. PLT/216

(57) ABSTRACT

A new Eucalyptus grandis tree particularly distinguished by having a fast growth rate, average frost tolerance, and good coppicing properties, is disclosed.

### ACKNOWLEDGEMENT OF FEDERAL RESEARCH SUPPORT

This invention was supported in part by funds from the U.S. Government pursuant to Grant No. FLA-FOR-04168 from USDA CSREES. The U.S. Government therefore has certain rights in the invention.

### LATIN NAME OF THE GENUS AND SPECIES OF THE PLANT CLAIMED

Eucalyptus grandis

### VARIETY DENOMINATION

‘G5’

### BACKGROUND OF THE INVENTION

The present invention comprises a new and distinct variety of Eucalyptus, botanically known as Eucalyptus grandis, and hereinafter referred to by the variety name ‘G5’. It is characterized by having a fast growth rate, average frost tolerance, and good coppicing properties when grown under normal forestry practices in southern Florida.

### SUMMARY OF THE INVENTION

‘G5’ originated from over 1,600 seedlings of Eucalyptus grandis. ‘G5’ is the result of open-pollination in a fourth-generation seedling seed orchard at Palmdale, Fla. The female (seed) parent of ‘G5’ is the proprietary Eucalyptus grandis seedling selection with the identification number ‘3242’, not patented, characterized by its fast growth and average frost tolerance. The male (pollen) parent of ‘G5’ is unknown. The maternal parent (‘3242’) originated from open-pollination. The female (seed) parent of ‘3242’ is the proprietary Eucalyptus grandis seedling selection with the identification number ‘1065’, not patented, characterized by its good growth and acceptable frost tolerance. The male (pollen) parent of ‘3242’ is unknown. ‘G5’ was selected as a single clone from the progeny of the above stated open-pollination during March 2004.

‘G5’ was first propagated in June of 2008 in Apopka, Fla. through cuttings and in vitro propagation. Asexually propagated trees of ‘G5’ have remained true to the original selected tree, and all characteristics of the tree have been transmitted and retained through three successive asexual vegetative generations.

Plant Breeder's Rights for this cultivar have not been applied for. ‘G5’ has not been made publicly available more than one year prior to the filing of this application.

‘G5’ differs from the female parent ‘3242’ by having faster growth, better frost tolerance, and good coppicing properties.

Commercial varieties: E.nergy G1, G2, G3, and G4 may be compared with ‘G5’.

### BRIEF DESCRIPTION OF THE DRAWINGS

This new eucalyptus tree is illustrated by the accompanying photographs which show the tree's form and foliage. The colors shown are as true as can be reasonably obtained by conventional photographic procedures. The photographs are of trees approximately 2 to 3.5-years old.

FIG. 1. Shows overall plant habit including foliage of a 3.5-year-old tree.

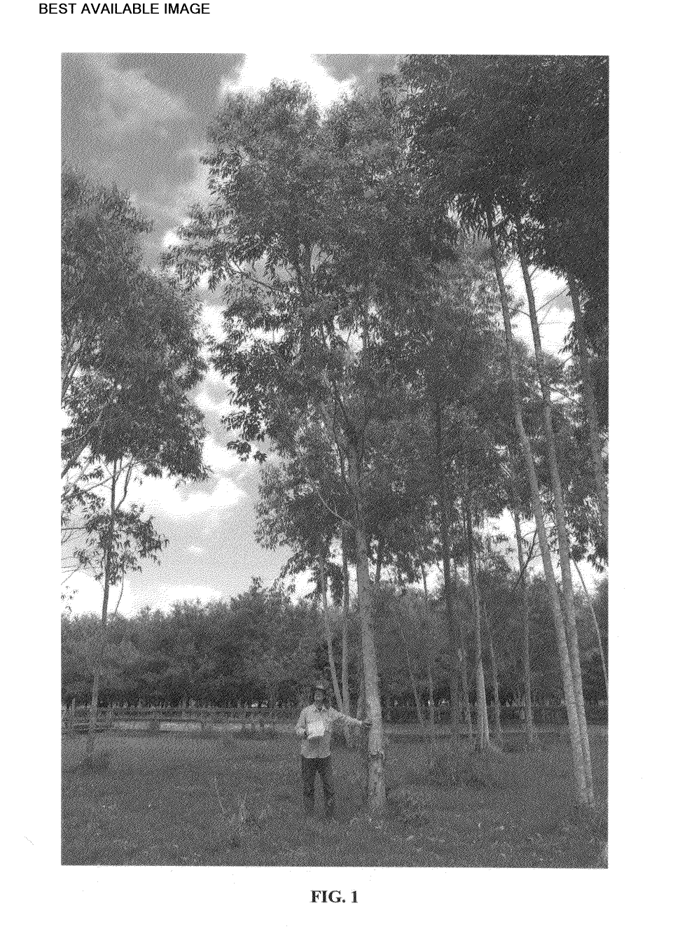

FIG. 2. Shows overall plant habit including foliage of 2-year-old trees.

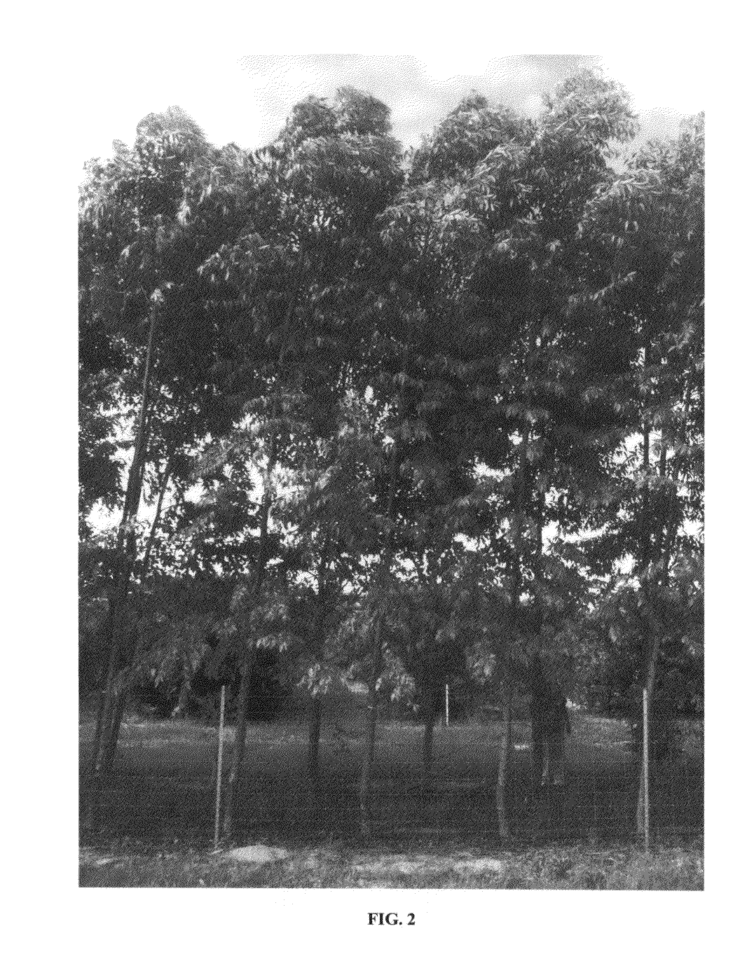

FIG. 3. Shows a close-up of the bark of a 3.5-year-old tree.

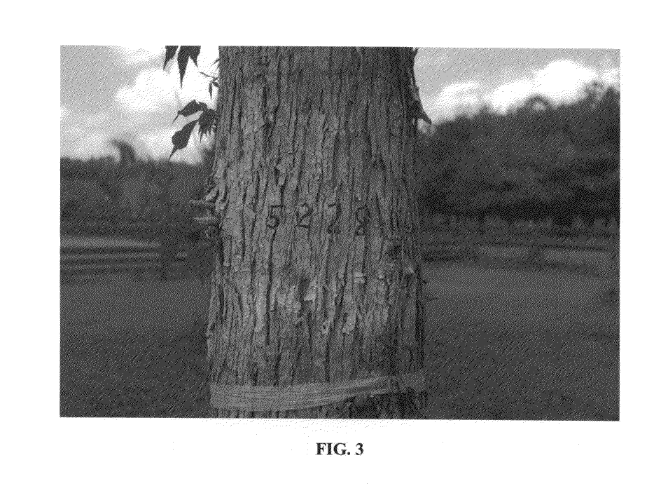

FIG. 4. Shows a close-up of the bark of a 2-year-old tree.

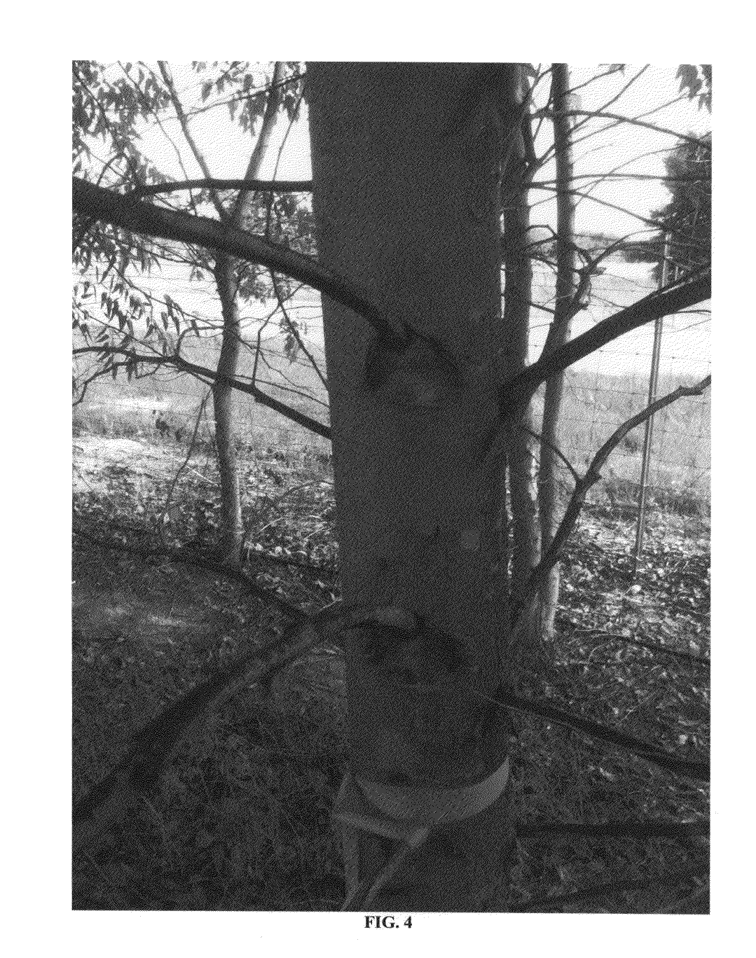

FIG. 5. Shows upper branches of a 3.5-year-old tree.

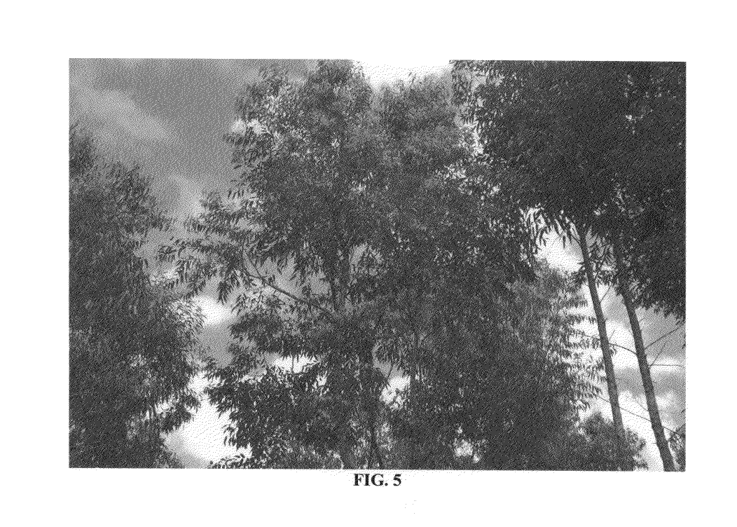

FIG. 6. Shows mature leaves of a 2-year-old tree.

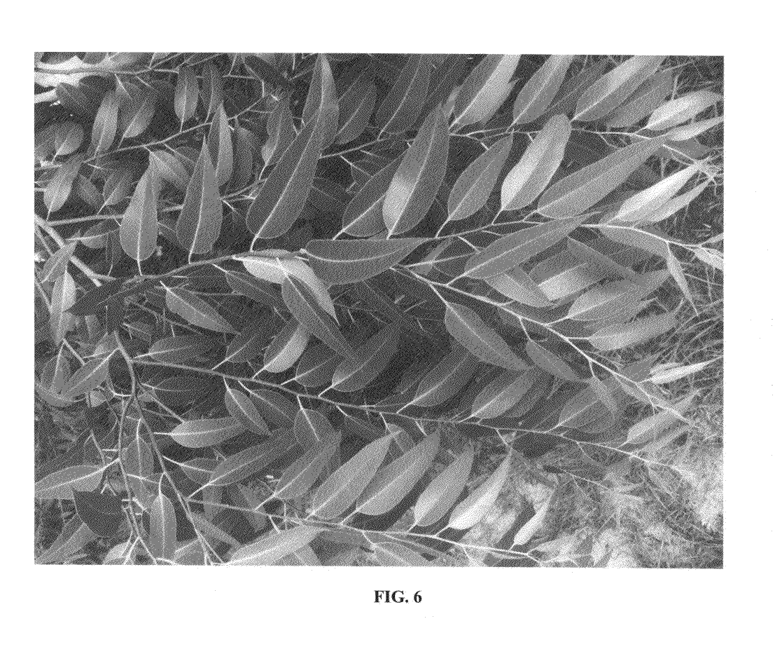

FIG. 7. Shows a close-up of mature leaves of a 2-year-old tree.

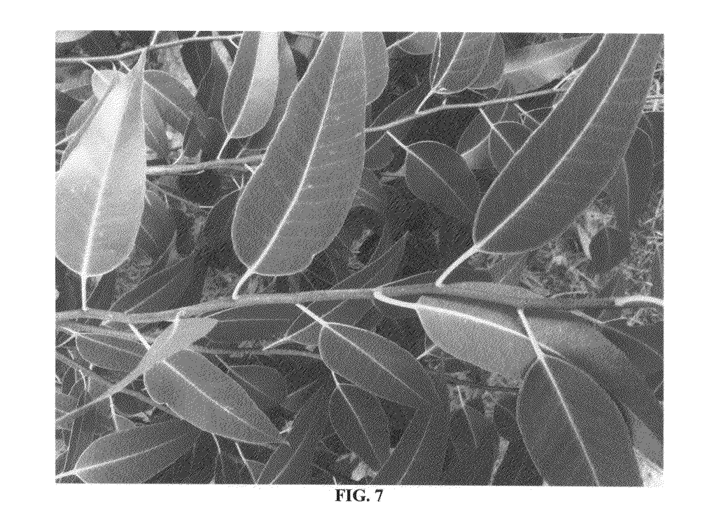

### DETAILED BOTANICAL DESCRIPTION

The following detailed description sets forth the distinctive characteristics of ‘G5’. The present botanical description is that of 2-year-old trees growing near Winter Garden, Fla. The colors (except those in common terms) are described from R.H.S. Colour Chart published by The Royal Horticultural Society in London (second edition), in association with the Flower Council of Holland.
Phenotypic description of Eucalyptus grandis var. ‘G5’
Classification:
   Botanical.—Eucalyptus grandis var. ‘G5’.
   Common name.—Eucalyptus.
Parentage:
   Female parent.—‘3242’, not patented
   Male parent.—Unknown.
Tree:
   Size.—Height — ˜10 m, Spread — ˜3 m.
   Vigor.—Average.
   Density.—Open.
   Form (overall shape of tree).—Good.
   Growth habit (current season).—Evergreen.
Trunk and branches:
   Trunk texture.—Smooth at top, rough at base.
Trunk bark color.—Mottled gray-brown.
Branch texture.—Glabrous.
   Branch color.—Gray to red.
Leaves:
   Size (lamina average).—Length — 10-14 cm, Width 2.0-3.5 cm.
   Type.—Simple, petiolate, alternate, persistent.
   Shape.—Lanceolate, slightly sickle shaped, slightly off-center midrib.
   Apex.—Acuminate.
   Base.—Acute, can be slightly unequilateral.
   Margin.—Smooth.
   Surface texture.—Upper — glabrous, Lower — glabrous.
   Color.—Upper — RHS 132B (Strong green), Lower — RHS 124B (Light green).
   Petiole.—Shape: Slightly flattened Length: 1.5-2.5 cm Width: 1-2 mm Color: RHS 11D (Pale yellow) Texture: Smooth.
Flowers: No flowering observed
Reproductive organs: No organs observed
Fruit/seed (if produced): No fruit/seed observed
Resistance/tolerance to diseases/pests: No unusual susceptibilities noted

What is claimed is:

1. A new and distinct variety of Eucalyptus grandis tree as shown and described herein.
